(19) United States
(12) Patent Application Publication
(10) Pub. No.: US 2025/0332343 A1
(43) Pub. Date: Oct. 30, 2025

(54) HANDLES FOR MEDICAL DEVICES

(71) Applicant: Boston Scientific Medical Device Limited, Galway (IE)

(72) Inventors: Nachiket GOLE, Pune (IN); Rajivkumar SINGH, Pune (IN)

(73) Assignee: Boston Scientific Medical Device Limited, Galway (IE)

(21) Appl. No.: 19/187,137

(22) Filed: Apr. 23, 2025

Related U.S. Application Data
(60) Provisional application No. 63/638,675, filed on Apr. 25, 2024.

(51) Int. Cl. A61M 5/158

(52) U.S. Cl. CPC A61M 5/158; A61M 2209/00

(57) ABSTRACT

A medical device handle may include a distal body portion, a proximal body portion, and a pair of arms, each arm of the pair of arms including a distal arm portion hingedly coupled to the distal body portion and a proximal arm portion hingedly coupled to the distal arm portion and coupled to the proximal body portion. Movement of the distal body portion relative to the proximal body portion may cause the handle to transition from a first configuration to a second configuration.

### CROSS-REFERENCE TO RELATED APPLICATION(S)

This application claims the benefit of priority under 35 U.S.C. § 119 from U.S. Provisional Application No. 63/638,675, filed on Apr. 25, 2024, which is incorporated by reference herein in its entirety.

### TECHNICAL FIELD

Various aspects of this disclosure relate generally to handles for medical devices. In particular, the disclosure pertains to handles with living hinges for injection needles.

### BACKGROUND

Injection needles may include a handle for gripping by an operator and a shaft for insertion into a body of a subject (e.g., via an insertion device such as a scope). A lumen of the shaft may receive a needle that may be selectively exposed or concealed relative to a distal tip of the shaft (e.g., a sheath). The needle may define a lumen through which a fluid, such as contrasting agent, saline, or another agent (e.g., a lifting agent) may be injected. The needle may have a distal opening that may be used to deliver the fluid to a treatment site within a body of the subject. The handle may include components for controlling the needle (e.g., advancing and retracting the needle), as well as components for coupling to a source of fluid.

### SUMMARY

Each of the aspects disclosed herein may include one or more of the features described in connection with any of the other disclosed aspects. Aspects of the disclosure may relate to handles for medical devices, such as injection needles. The handles may have living hinges to enable extension/retraction and/or locking of the needle and may have elements that are integrally formed of a single piece of material.

For example, a medical device handle may include a distal body portion, a proximal body portion, and a pair of arms, each arm of the pair of arms including a distal arm portion hingedly coupled to the distal body portion and a proximal arm portion hingedly coupled to the distal arm portion and coupled to the proximal body portion. Movement of the distal body portion relative to the proximal body portion may cause the handle to transition from a first configuration to a second configuration.

Any of the systems, devices, and methods disclosed herein may include any of the following features. A body of the medical device handle includes the distal body portion, the proximal body portion, and the pair of arms. The body may include a gap between the distal body portion and the proximal body portion and between arms of the pair of arms. The proximal body portion may be fixedly coupled to a member extending through the shaft. Transitioning from the first configuration to the second configuration may cause the shaft to move relative to the member. The member may be a needle, and a distalmost end of the needle may be proximal of a distalmost end of the shaft in the first configuration. The distalmost end of the needle may be distal to the distalmost end of the shaft in the second configuration. The proximal body portion, the distal body portion, and the arms may be integrally formed. The medical device handle may further include a removable retention mechanism configured to inhibit the distal body portion from moving relative to the proximal body portion. Each of the proximal arm portions may be hingedly coupled to a respective distal arm portion by a living hinge. Each of the distal arm portions may be hingedly coupled to the distal body portion by a living hinge. Each of the proximal arm portions may be fixedly coupled to the proximal body portion. In the first configuration, each of the proximal arm portions may have a first angle with respect to a respective distal arm portion, and in the second configuration, each of the proximal arm portions may have a second angle with respect to the respective distal arm portion. The second angle may be smaller than the first angle. In the first configuration, an end of the distal arm portion coupled to the distal body portion may be distal to an end of the distal arm portion coupled to the proximal arm portion. In the second configuration, the end of the distal arm portion coupled to the distal body portion may be proximal of the end of the distal arm portion coupled to the proximal arm portion. The medical device may further include a lock mechanism configured to retain the handle in the second configuration.

This disclosure further includes a medical device handle including a distal body portion fixedly coupled to a shaft, a proximal body portion fixedly coupled to a member extending through the shaft, and a pair of arms extending between the distal body portion and the proximal body portion, each arm of the pair of arms including a distal arm portion and a proximal arm portion hingedly coupled to the distal arm portion. Movement of the distal body portion relative to the proximal body portion may cause the shaft to move relative to the member.

Any of the systems, devices, and methods disclosed herein may include any of the following features. The distal body portion, the proximal body portion, and the pair of arms may be integrally formed with one another. Each of the proximal arm portions may be hingedly coupled to a respective distal arm portion by a living hinge.

This disclosure further includes a medical device handle including a distal body portion, a proximal body portion, and a pair of arms extending between the distal body portion and the proximal body portion, each arm of the pair of arms may include a distal arm portion having a first lock member and a proximal arm portion hingedly coupled to the distal arm portion and having a second lock member. A configuration of the handle, the first lock member may engage the second lock member.

Any of the systems, devices, and methods disclosed herein may include any of the following features. The distal body portion, the proximal body portion, and the pair of arms may be integrally formed with one another.

### BRIEF DESCRIPTION OF THE DRAWINGS

The accompanying drawings, which are incorporated in and constitute a part of this specification, illustrate aspects this disclosure and together with the description, serve to explain the principles of the disclosure.

FIG. 1 depicts an exemplary medical device.

FIGS. 2A-2B depict a portion of the exemplary medical device in a first configuration.

FIGS. 3A-3B depict the portion of the exemplary medical device in a second configuration.

### DETAILED DESCRIPTION

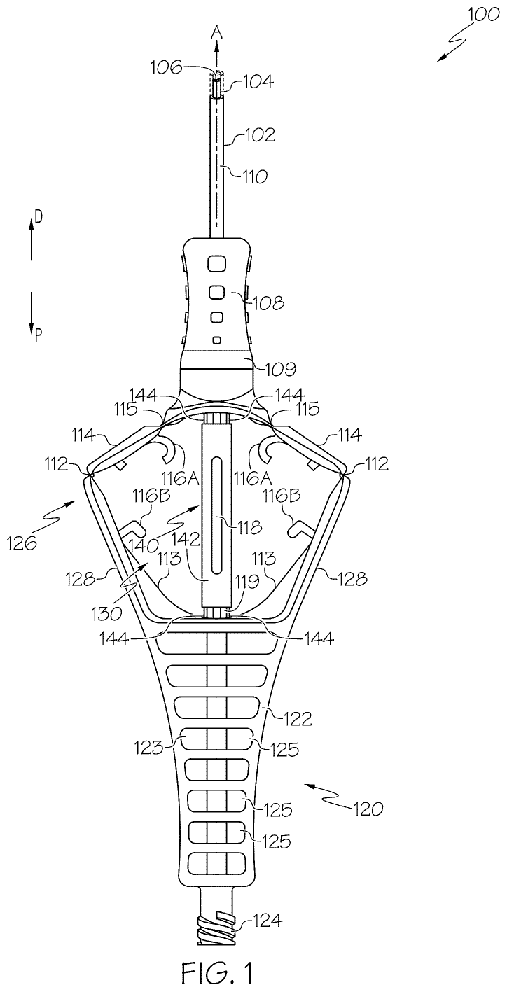

It may be understood that both the foregoing general description and the following detailed description are exemplary and explanatory only and are not restrictive of the invention, as claimed. As used herein, the terms “comprises,” “comprising,” “has,” “having,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator, and the term “proximal” refers to a direction toward an operator. FIG. 1 includes arrows labeled “P” and “D” to indicate proximal and distal directions, respectively. The term “exemplary” is used in the sense of “example,” rather than “ideal.” The term “approximately,” or like terms (e.g., “substantially”), includes values +/−10% of a stated value. Unless otherwise stated, ranges disclosed herein include the end points of the ranges.

Medical devices, such as injection needles, may include a handle to be gripped by an operator and a shaft distally extending from the handle. The shaft may be configured to be inserted into a body of a subject (e.g., via an introduction device such as an endoscope or other type of scope). A feature or element at the distal end of the shaft (e.g., a needle) may be operable to perform a procedure at a treatment site within the body of the subject. The handle may include members connected by living hinges that transition the medical device between two configurations. The members may include proximal arm portions and distal arm portions. The movement of two flexing arms may cause extension and/or retraction of the shaft relative to an injection needle or other instrument. The handle may be adjusted between an unlocked and a locked position. For example, an operator may squeeze the arms to connect pairs of snap locks. A body of the handle may be molded from a single piece of material, such as a polymer. For example, the entire handle assembly may be manufactured using an injection molding technique.

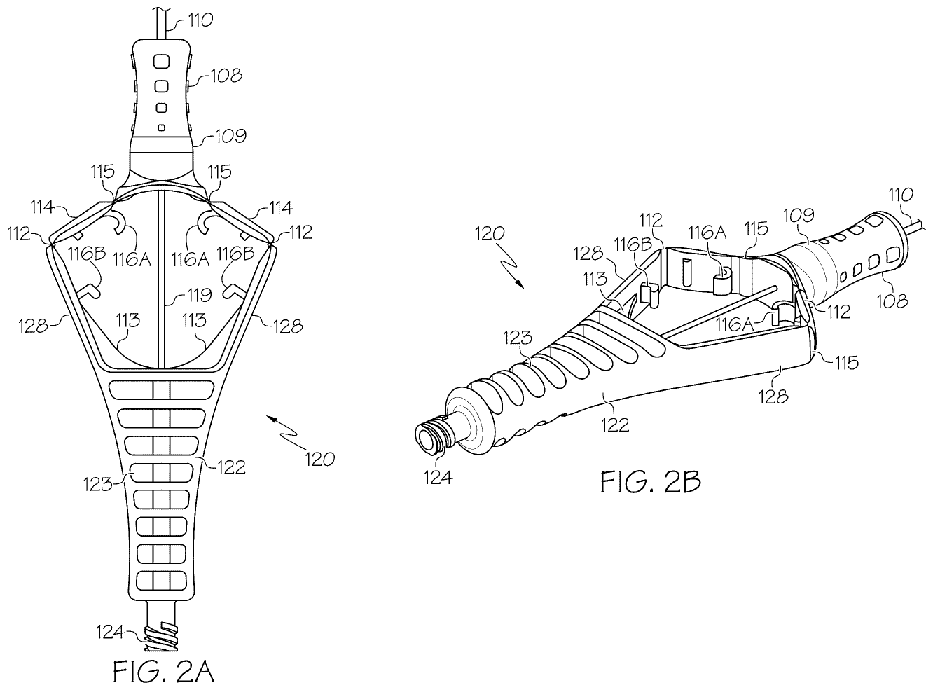

FIGS. 1, 2A, and 2B shows a medical device 100 in a first (e.g., relaxed or delivery) configuration. Medical device 100 may include a shaft 110 and a handle 120. Shaft 110 may have a distal end 102. A portion of distal end 102 of shaft 110 is shown in broken lines to indicate transparency and to reveal aspects that are internal to shaft 102. A needle 104 may extend through shaft 110, to distal end 102. Shaft 110 may be movable with respect to needle 104, such that relative movement between shaft 110 and needle 104 (e.g., proximal movement of shaft 110 relative to needle 104 or distal movement of needle 104 relative to shaft 110) may expose needle 104 (e.g., to a position distally beyond) a distal end of shaft 110. Similarly, relative movement between shaft 110 and needle 104 (e.g., distal movement of shaft 110 relative to needle 104 or proximal movement of needle 104 relative to shaft 110) may conceal or sheath needle 104. Relative movement of the shaft 110 and/or needle 104 may be on the order of approximately 15-20 mm, though this is only exemplary. In the concealed configuration, a distalmost end of needle 104 may be proximal of a distalmost end of shaft 110.

Needle 104 may have a lumen 106 extending therethrough. As discussed below, a portion of handle 120 may be fixedly coupled to needle 104 or another element (e.g., end effector). Although needle 104 is described herein, it will be appreciated that medical device 100 may include other types of features at its distal tip (e.g., basket, snare, forceps, stapler, etc.), and medical device 100 is not limited by a type of instrument at its distal tip.

Handle 120 may have a body 122. Body 122 may include a proximal (e.g., second) gripping portion 123. Handle 120 may include a gripping portion 123. Gripping portion 123 may be a proximal portion of handle 120 (e.g., of body 122) that is configured to be held by an operator during a medical procedure. In some aspects, gripping portion 123 may include cut-away portions 125. Cut-away portions 125 of gripping portion 123 are depicted as approximately ovular, but any suitable shape or quantity of cut-away portions 125 may be implemented with the exemplary embodiments. Cut-away portions may have approximately similar shapes, or may be of varying shape. Cut-away portions 125 of gripping portion 123 may increase ergonomics (e.g., grip) of gripping portion 123 and/or offer reductions in material (e.g., polymer) usage. In some aspects, cut away portions 125 may not be present. In some aspects, gripping portion 123 may feature varying thickness along the longitudinal axis such that the ergonomic profile of gripping portion 123 is enhanced. For example, gripping portion 123 may taper laterally outwardly moving in a distal direction.

Body 122 may also include two arms 126. Each arm 126 may include a proximal portion 128 and a distal portion 114. Proximal portions 128 may extend distally from gripping portion 123. For example, proximal ends of each proximal portion 128 may be fixedly coupled to opposite, laterally outer sides of gripping portion 123, at a distalmost end of gripping portion 123. Proximal portions 128 may extend laterally outward, away from a central longitudinal axis A of device 100. For example, a smooth curve or line may extend along each of proximal portions 128 and corresponding sides of gripping portion 123. As discussed in further detail below, proximal portions 128 may be integrally formed with gripping portion 123, from a single piece of material.

Proximal portions 128 may be coupled to distal portions 114 via hinges 112. In some examples, hinges 112 may be living hinges. For example, hinges 112 may be flexible portions of arms 126 that may deform in response to an applied force. Hinges 112 may have a reduced cross-sectional thickness as compared to proximal portions 128 and distal portions 114. The reduced cross-sectional thickness of hinges 112 may enable arms 126 to bend about hinges 112. In alternatives, hinges 112 may not be living hinges and may include pins, rivets, or the like for coupling proximal portions 128 to distal portions 114.

Distal portions 114 may extend laterally inward, toward longitudinal axis A, moving in a distal direction. Compared with proximal portions 128, distal portions 114 may have a shorter length, although such a configuration is merely exemplary. In some examples, in the first configuration of FIG. 1, distal portions 114 may have an approximately 90 degree angle with respect to proximal portions 128, although such an angle is merely exemplary. In examples, in the first configuration of FIG. 1, distal portions 114 may have an obtuse angle (which may be less than 180 degrees) with respect to proximal portions 128.

Resilient members 113 (e.g., support beams) may extend between each proximal portion 128 and a distal end of gripping portion 123. Resilient members 113 may provide support to proximal portions 128, inhibiting proximal portions 128 from bending with respect to gripping portion 123. In alternatives, resilient members 113 may be flexible beams or other structures that may bias arms 126 toward a certain orientation.

Handle 120 (e.g., body 122) may include a distal (e.g., first) gripping portion 109 Distalmost ends of distal portions 114 may be coupled to a proximalmost end of gripping portion 109. Gripping portion 109 may be connected to distal portions 114 via hinges 115 (e.g., living hinges having any of the features of hinges 112). In some examples, gripping portion 109 may have an approximately tubular shape. Side surfaces of gripping portion 109 may curve radially inward, such that gripping portion 109 has a concave shape to assist in gripping of gripping portion 109. Gripping portion 109 may include gripping elements 108. An operator may grip handle 120 via gripping portion 109 and gripping elements 108. Gripping elements 108 may include a plurality of ridges, a flexible material (e.g., an overmolded material), recesses, or other similar features.

A proximalmost end of shaft 110 may be fixedly coupled to gripping portion 109. For example, a proximalmost end of shaft 110 may be received within a lumen of gripping portion 109 and may be fixedly coupled thereto. The proximalmost end of shaft 110 may be within or adjacent to (e.g., distally adjacent to) gripping portion 109.

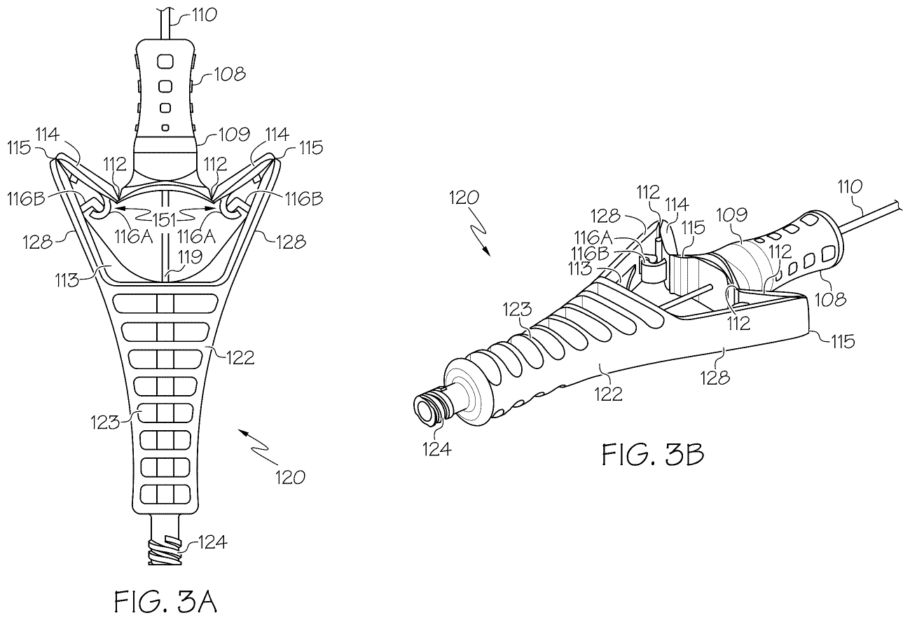

A tube 119 (also shown in FIGS. 2A-3B) may be coupled to needle 104. For example, tube 119 may be integrally formed with needle 104, and needle 104 may refer to a distal portion of tube 119. In alternatives, tube 119 may be a shaft, wire, cable, or other actuation mechanism (e.g., where an end effector of device 100 is not a needle). Tube 119 and/or needle 104 may extend through a length of shaft 110. Tube 119 may extend proximally of the proximalmost end of shaft 110. Tube 119 may extend through the lumen of gripping portion, through gap 130 between arms 126, and into proximal gripping portion 123. Proximal gripping portion 123 may define a lumen for receiving and securing tube 119. Tube 119 may be fixedly coupled to a proximal portion of handle 120.

Gap 130 may be formed between arms 126 and between proximal gripping portion 123 and distal gripping portion 109. Gap 130 may be an open space, such that body 122 has a hole or opening in it that defines gap 130. A size and shape of gap 130 may change as distal portions 114 of arms 126 and distal gripping portion 109 move relative to proximal portions 128 of arms 126 and proximal gripping portion 123. A central longitudinal axis of distal gripping portion 109 may be coaxial with a central longitudinal axis of proximal gripping portion 123 and longitudinal axis A of device 100. Arms 126 may be symmetrical about longitudinal axis A.

Body 122 may also include one or more locking mechanisms 151 (see FIG. 3A). For example, body 122 may include two locking mechanisms 151. Each of locking mechanisms 151 may include a distal member 116A and a proximal member 116B. Distal member 116A may extend from distal portion 114 of arm 126, and proximal member 116B may extend from proximal portion 128 of arm 126. Distal member 116A and proximal member 116B may be a hook and a tab, respectively, or any suitable locking mechanism. For example, distal member 116A may have a curved, hook shape. Proximal member 116B may have an angled shape, with a first portion that extends approximately 90 degrees from an inner surface of proximal portion 128 of arm 126 and a second portion that extends approximately 90 degrees from the first portion, approximately parallel to the inner surface of proximal portion 128 and in a proximal direction.

As shown in FIG. 3A and described below, distal member 116A may mate with proximal member 116B when hinges 112 bend and bring distal member 116A and proximal member 116B into mating range. In the configurations of FIGS. 1-2B, locking mechanisms 151 may be unlocked.

One of ordinary skill in the art will appreciate that the positions of distal member 116A and proximal member 116B are only exemplary. For example, distal member 116A and proximal member 116B may have their respective positions switched without deviation from the scope the exemplary embodiments. In some aspects, additional locks may be included in addition to locking mechanisms 151. For example, additional locking mechanisms may correspond with a partially deployed configuration of needle 104. Locking mechanisms 151 may be used to selectively retain shaft 110 in a desired position relative to needle 104.

Handle 120 (e.g., body 122) may include a fluid port 124. Fluid port 124 may be used to attach handle 120 to a fluid source, such as a syringe. In some examples, fluid port 124 may include a Luer mating. Fluid port 124 is depicted with a screw coupler. Various neck widths and thread finishes of fluid port 124 are possible such that handle 120 is compatible with a variety of fluid sources. Fluid port 124 may be in fluid communication with a lumen (e.g., lumen 106) of tube 119, such that fluid from fluid port 124 may flow into lumen 106.

Handle 120 may include a retention mechanism 140 (FIG. 1), which may include a body 142 and a tab 118. Retention mechanism 140 may inhibit actuation of handle 120 and be removable by an operator before using handle 120 prior to or during a medical procedure. Retention mechanism 140 may inhibit unintentional deployment of needle 104 during shipping of handle 120 and during pre-procedure preparation by an operator. Retention mechanism 140 may inhibit distal portion 114 of arm 126 from bending about hinge 112 relative to proximal portion 128 of arm 126. Actuation of handle 120 is described in further detail below. Retention mechanism may extend between proximal gripping portion 123 and distal gripping portion 109, preventing longitudinal movement of distal gripping portion 109 relative to proximal gripping portion 123.

Body 142 may extend partially around tube 119. For example, body 142 may have an approximately tubular shape, with one or more via one or more openings or perforations formed across the longitudinal length of body 142. The openings may have a larger width than a width of tube 119. One or more strands or beams 144 may extend between body 142 and gripping portions 109, 123. In examples, retention mechanism 140 may be of the same unibody structure as the other components of body 122.

Retention mechanism 140 may be removed by an operator pulling and/or twisting retention mechanism 140 so as to break beams 144. FIGS. 2A and 2B show handle 120 with retention mechanism 140 removed. Body 142 may thus be removed from tube 119 via the opening extending longitudinally along body 142. For example, an operator may grip tab 118 to remove retention mechanism 140. Tab 118 may have a semicircular or any other suitable shape to facilitate gripping by the operator. In some aspects, retention mechanism 140 may not be present. In the configuration of FIGS. 2A and 2B, distal gripping portion 109 and distal portion 114 of arm 126 may be movable relative to proximal gripping portion 123 and proximal portion 128 of arm 126, as described below.

Body 122 may be integrally formed from a single piece of material (e.g., a unibody structure), such as an injection-molded polymer body. For example, port 124, proximal gripping portion 123, arms 126, distal gripping portion 109, and/or retention mechanism 140 may be formed of a single, unitary piece of material. The unibody structure of body 122 may offer advantages in the manufacturing process, such as reduced assembly time and reduced part complexity (e.g., living hinges 112 and 115).

FIGS. 3A and 3B show handle 120 in a second, actuated configuration. To transition from the first configuration of FIGS. 1-2B to the second configuration of FIGS. 3A-3B, an operator may move distal gripping portion 109 proximally, toward proximal gripping portion 123. Proximal movement of gripping portion 109 may cause a portion of distal portion 114 coupled to distal gripping portion 109 to also move proximally. An angle between gripping portion 109 and distal portion 114 may change (e.g., decrease, become smaller, or move from obtuse to acute) as distal portion 114 of arm 126 moves. Arm 126 may also bend at hinge 112. Thus, an angle between gripping portion 109 and distal portion 114 may be smaller in the second configuration than in the first configuration.

As discussed above, proximal portion 128 may be fixed, such that it has a same configuration in the first configuration and the second configuration of handle 120. An angle between distal portion 114 of arm 126 and proximal portion 128 of arm 126 may change (e.g., decrease, become smaller, or move from obtuse to acute). In some examples, as shown in the FIGS. 3A and 3B, in the second configuration, and end of distal portion 114 that is coupled to distal gripping portion 109 (via hinge 115) may be proximal to an end of distal portion 114 that is coupled to proximal portion 128 (via hinge 112). In contrast, in the first configuration, as shown in FIGS. 1-2B, an end of distal portion 114 that is coupled to distal gripping portion 109 may be distal to an end of distal portion 114 that is coupled to proximal portion 128.

Thusly, distal gripping portion 109 may move proximally relative to proximal gripping portion 123. As discussed above, distal gripping portion 109 may be fixedly coupled to shaft 110, and proximal gripping portion 123 may be fixedly coupled to needle 104 (directly or via tube 119). Thus, shaft 110 may move proximally relative to needle 104, and a distal portion of needle 104 may be unsheathed/exposed (actuated).

In alternatives, the operator may move proximal gripping portion 123 distally relative to distal gripping portion 109 in order to achieve the configuration of FIGS. 3A and 3B. In such situations, needle 104 may move distally to unsheath/expose a distal portion of needle 104, rather than shaft 110 moving proximally. In a further alternative, a combination of moving proximal gripping portion 123 and distal gripping portion 109 may occur. The relative movement between proximal gripping portion 123 and distal gripping portion 109 may actuate needle 104, thereby exposing or unsheathing needle 104.

In FIGS. 3A-3B, handle 120 is depicted in a locked configuration. In the locked configuration, locking mechanisms 151 may be engaged. Handle 120 may be retained in the second configuration when force is removed from handle 120 (e.g., when a user lets go of handle 120 or ceases moving distal and/or proximal gripping portions 109, 123) because distal members 116A and proximal members 116B have engaged one another. To transition handle 120 from the second configuration to the first configuration, an operator may apply move distal gripping portion 109 further proximally (or proximal gripping portion 123 further distally) to disengage locking mechanisms 151. Alternatively, the operator may apply sufficient distal force on distal gripping portion 109 and/or sufficient proximal force on proximal gripping portion 123 in order to disengage locking mechanisms 151.

Handle 120 may have one or more resilient features to bias handle 120 to the first configuration. For example, hinges 112 and/or 115 may be biased to the first configuration. Handle 120 may additionally or alternatively include one or more resilient members (e.g., springs) to bias handle 120 to the first configuration. Such features may serve as a safety feature of handle 120, such that needle 104 defaults to an unexposed position when an operator has not engaged the locking mechanism 151. Additionally, such resilient features may provide a biasing force when handle 120 is in the locked position such that distal members 116A and proximal members 116B remain in firm contact with one another during a medical procedure.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Additionally, a variety of elements from each of these embodiments can be combined to achieve a same or similar result as one or more of the disclosed embodiments. Accordingly, the invention is not to be considered as limited by the foregoing description.

We claim:

1. A medical device handle comprising:
a distal body portion;
a proximal body portion; and
a pair of arms, each arm of the pair of arms comprising:
a distal arm portion hingedly coupled to the distal body portion; and
a proximal arm portion hingedly coupled to the distal arm portion and coupled to the proximal body portion;
wherein movement of the distal body portion relative to the proximal body portion causes the handle to transition from a first configuration to a second configuration.

2. The medical device handle of claim 1, wherein a body of the medical device handle includes the distal body portion, the proximal body portion, and the pair of arms, and wherein the body includes a gap between the distal body portion and the proximal body portion and between arms of the pair of arms.

3. The medical device handle of claim 1, wherein the distal body portion is fixedly coupled to a shaft, wherein the proximal body portion is fixedly coupled to a member extending through the shaft, and wherein transitioning from the first configuration to the second configuration causes the shaft to move relative to the member.

4. The medical device handle of claim 3, wherein the member is a needle, and wherein a distalmost end of the needle is proximal of a distalmost end of the shaft in the first configuration.

5. The medical device handle of claim 4, wherein the distalmost end of the needle is distal to the distalmost end of the shaft in the second configuration.

6. The medical device handle of claim 1, wherein the proximal body portion, the distal body portion, and the arms are integrally formed.

7. The medical device handle of claim 1, further including a removable retention mechanism configured to inhibit the distal body portion from moving relative to the proximal body portion.

8. The medical device handle of claim 1, wherein each of the proximal arm portions is hingedly coupled to a respective distal arm portion by a living hinge.

9. The medical device handle of claim 8, wherein each of the distal arm portions is hingedly coupled to the distal body portion by a living hinge.

10. The medical device handle of claim 9, wherein each of the proximal arm portions is fixedly coupled to the proximal body portion.

11. The medical device handle of claim 2, wherein, in the first configuration, each of the proximal arm portions has a first angle with respect to a respective distal arm portion, and wherein, in the second configuration, each of the proximal arm portions has a second angle with respect to the respective distal arm portion.

12. The medical device handle of claim 22, wherein the second angle is smaller than the first angle.

13. The medical device handle of claim 2, wherein, in the first configuration, an end of the distal arm portion coupled to the distal body portion is distal to an end of the distal arm portion coupled to the proximal arm portion.

14. The medical device handle of claim 13, wherein, in the second configuration, the end of the distal arm portion coupled to the distal body portion is proximal of the end of the distal arm portion coupled to the proximal arm portion.

15. The medical device handle of claim 1, further comprising: a lock mechanism configured to retain the handle in the second configuration.

16. A medical device handle comprising:
a distal body portion fixedly coupled to a shaft;
a proximal body portion fixedly coupled to a member extending through the shaft; and
a pair of arms extending between the distal body portion and the proximal body portion, each arm of the pair of arms comprising:
a distal arm portion; and
a proximal arm portion hingedly coupled to the distal arm portion;
wherein movement of the distal body portion relative to the proximal body portion causes the shaft to move relative to the member.

17. The medical device handle of claim 16, wherein the distal body portion, the proximal body portion, and the pair of arms are integrally formed with one another.

18. The medical device handle of claim 16, wherein each of the proximal arm portions are hingedly coupled to a respective distal arm portion by a living hinge.

19. A medical device handle comprising:
a distal body portion;
a proximal body portion; and
a pair of arms extending between the distal body portion and the proximal body portion, each arm of the pair of arms comprising:
a distal arm portion having a first lock member; and
a proximal arm portion hingedly coupled to the distal arm portion and having a second lock member;
wherein, in a configuration of the handle, the first lock member engages the second lock member.

20. The medical device handle of claim 19, wherein the distal body portion, the proximal body portion, and the pair of arms are integrally formed with one another.
